(19) United States
(12) Patent Application Publication
(10) Pub. No.: US 2009/0118745 A1
(43) Pub. Date: May 7, 2009

(54) PATENT FORAMEN OVALE CLOSURE APPARATUS AND METHOD

(75) Inventor: Ram H. Paul, JR., Bloomington, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/935,500

(22) Filed: Nov. 6, 2007

(51) Int. Cl. A61B 17/08

(52) U.S. Cl. 606/151; 606/216

(57) ABSTRACT

An apparatus for closure of a patent foramen ovale of a patient comprises a frame member defining respective first, second and third frame segments, and a growth and/or clot promoting material positioned on at least one of the segments. The frame member is alignable in a generally elongated first position for deployment via a delivery catheter, and is elastically movable therefrom to a compressed second position when the frame member is deployed across the foramen ovale. The frame segments are structured and arranged such that upon deployment of the apparatus across the foramen ovale, the first frame segment is disposed distal of the atrial septum, the second frame segment is disposed intermediate the atrial septum and the tissue flap, and the third frame segment is disposed proximal of the tissue flap.

### BACKGROUND OF THE INVENTION

1. Technical Field

The present invention relates to a percutaneously-introduced apparatus for closure of a patent foramen ovale (PFO) in a patient, and to a method for closure of a patent foramen ovale utilizing the percutaneously-introduced apparatus.

2. Background Information

In the fetal heart, there is a small communication, referred to as the foramen ovale, in the septum between the right and left atria. In the unborn fetus, this communication allows blood to bypass the lungs. Fetal blood is oxygenated by the lungs of the mother. This communication normally closes within the first year after birth, and oxygenation is carried out through the baby's own lungs. Although the remnant of the opening remains in the septum after birth, the remnant normally does not allow passage of blood.

In some cases, however, this opening (the foramen ovale) remains patent and the baby's oxygenated blood is diluted by un-oxygenated venous blood. Babies with this condition often have very little energy, are cyanotic (blue coloration), and do not progress well after birth. In order to repair this defect, the opening can be closed by surgical methods, or by newer percutaneous methods.

In recent years, physicians have also discovered that in a large percentage of adults, estimated at about 30%, the foramen ovale has not completely sealed, and remains as a small patent foramen ovale. In these adults, there is still some leakage across the septum through the remnant foramen ovale. Although such leakage is not always problematic, the leakage can be aggravated upon certain types of strain or valsalva. Intermittent leakage of blood through the PFO has been linked to migraine headaches and other maladies. In addition, a PFO is suspected as being a passageway for blood clots. Passage of clots through the opening can lead to a stroke or a transient ischemic attack (TIA).

The leaking, or patent, foramen ovale does not result from the same physiological structure as an atrial septal defect (ASD). An ASD is normally a definable hole that extends through the septum. Such holes can be occluded by passing known occluder devices through the hole, such that the devices anchor on each side of the septum to form a seal. Current devices that are commonly used for ASD repair include the Amplatzer ASD Occluder, available from AGA Medical, and the Gianturco occluder coils, available from Cook Incorporated.

Unlike the definable hole that forms an ASD, the foramen ovale is a small channel or slot-type structure that is defined by the septum and a flap that covers a part of the ovale. With a PFO, the septum and the flap normally overlap to a certain degree, and are not fused together as in the normal case. As a result, small amounts of blood may leak through a passageway that extends between the septum and the flap.

Currently available ASD repair devices are ill suited for repair of a PFO. As stated, ASD repair devices normally comprise an occluder-type structure that is extended through the septum hole that comprises the defect to seal the opening. However, with a PFO, the openings on each side of the septum are offset, and not in line with each other (i.e., not directly across from each other). The leakage path is under a flap, and through a narrow passageway, rather than a defined hole. Thus, it is not generally sufficient to merely provide a plug for a hole, as in conventional ASD repair.

Open heart surgical methods have been used for PFO repair. Such methods normally entail breaking open the chest cavity, and cutting into the heart muscle. The flap is then sutured or otherwise attached to the septum, in a manner such that the passageway is closed. Although generally effective for closing the PFO, such methods are intrusive, costly, and require an extended recovery period for the patient.

Recently, percutaneous methods have been developed for repair of a PFO. These methods involve utilizing conventional percutaneous entry techniques, such as the Seldinger technique, and passing a catheter through a vessel into the right atrium of the heart. One device used in such methods, known as the Amplatzer PFO Occluder, comprises a plug-like device formed of a self-expanding wire-mesh with double discs. This device contains inner polyester fabric patches that, along with the wire mesh, are intended to cause the formation and accumulation of a blood clot. The resulting blood clot is positioned to block the opening. Devices of this type are complex mechanically, require a high level of skill to insert properly, and rely on a clot to actually form the seal.

There exists a need for an apparatus for providing effective closure of a patent foramen ovale, which apparatus is suitable for percutaneous entry, is less complex mechanically and operationally when compared to prior art devices, and can be utilized for patent foramen ovale closure with minimal trauma to the patient.

### SUMMARY

The problems of the prior art are addressed by the present invention. In one form thereof, the invention comprises an apparatus for closure of a patent foramen ovale of a patient, the foramen ovale defined by an opening in the heart of the patient between the atrial septum and a tissue flap. The apparatus includes a frame member defining a plurality of frame segments alignable in a generally elongated first position, and movable therefrom to a longitudinally compressed second position. The frame segments are structured and arranged such that upon deployment of the apparatus across the foramen ovale in the generally elongated first position and movement therefrom to the compressed second position, a first frame segment is disposed distal of the atrial septum, a second frame segment is disposed intermediate the atrial septum and the tissue flap, and a third frame segment is disposed proximal of the tissue flap. At least one of the frame segments includes a cover material disposed at least partially thereover. The cover material is capable of promoting biological formation to effect a closure of the foramen ovale.

In another form thereof, the invention comprises an apparatus for closure of a patent foramen ovate of a patient. A frame member defines respective first, second and third frame segments. The frame member is alignable into a generally elongated first position, and movable therefrom to a longitudinally compressed second position. The frame member is formed from a composition having elasticity sufficient for moving the frame segments from the first position to the second position when the frame member is deployed across the foramen ovate. The frame segments are structured and arranged such that upon deployment of the apparatus across the foramen ovale in the generally elongated first position and movement therefrom to the compressed second position, the first frame segment is disposed distal of the atrial septum, the second frame segment is disposed intermediate the atrial septum and the tissue flap, and the third frame segment is disposed proximal of the tissue flap. The second frame segment includes a cover material disposed at least partially thereover, wherein the cover material is capable of promoting biological formation to effect a closure of the foramen ovale.

In yet another form thereof, the invention comprises a method for closure of a patent foramen ovale defined by an opening in the heart of the patient between the atrial septum and a tissue flap. A closure apparatus comprising a frame member defining a plurality of frame segments is provided. The frame segments are alignable into a generally elongated first position, and movable therefrom to a longitudinally compressed second position. The frame segments are structured and positioned such that upon deployment of the apparatus across the patent foramen ovate, a first frame segment is disposed distal of the atrial septum, a second frame segment is disposed intermediate the atrial septum and the tissue flap, and a third frame segment is disposed proximal of the tissue flap. At least one of the frame segments includes a cover material disposed at least partially thereover, which cover material is capable of promoting biological formation to effect a closure of the foramen ovale. The closure apparatus is loaded into a delivery sheath in the elongated position, and the delivery sheath and loaded closure apparatus are percutaneously introduced into the right atrium of the heart of a patient. The delivery sheath and loaded closure apparatus are introduced such that a distal tip of the delivery sheath is positioned distal of the atrial septum, and the first frame segment is deployed from the delivery sheath. The delivery sheath is withdrawn such that the distal tip is positioned intermediate the atrial septum and the tissue flap, and the second frame segment is deployed from the delivery sheath. The delivery sheath is further withdrawn such that the distal tip is positioned proximal of the tissue flap, and the third frame segment is deployed from the delivery sheath.

### BRIEF DESCRIPTION OF THE DRAWINGS

FIG. 1 is an illustration of the internal portions of the heart;

FIG. 1A is an enlarged view of a portion of FIG. 1, illustrating the location of a patent foramen ovale;

FIG. 2 is a side view of an apparatus for closure of a patent foramen ovale in an elongated condition;

FIG. 3 is a view of the apparatus of FIG. 2, in a partially closed condition;

FIGS. 4-6 illustrate views of the apparatus of FIG. 2 positioned across a patent foramen ovale, shown in various stages of closure; and

FIG. 7 is a side view of a delivery device showing the PFO closure apparatus in position for deployment.

### DESCRIPTION OF PREFERRED EMBODIMENTS

For purposes of promoting an understanding of the present invention, reference will now be made to the embodiments illustrated in the drawings, and specific language will be used to describe the same. It is nevertheless to be understood that no limitation of the scope of the invention is thereby intended. The figures are not all drawn to the same scale to avoid obscuring the details of the finer structures. The following detailed description of the preferred embodiments will make clear the preferred arrangement, size relationships and manner of using the components shown herein.

The present invention relates to an apparatus and method for patent foramen closure. In the following discussion, the terms “proximal” and “distal” will be used to describe the opposing axial ends of the apparatus, as well as the axial ends of various component features of the apparatus. The term “proximal” is used in its conventional sense to refer to the end of an apparatus (or component) that is closest to the operator during use of the apparatus. The term “distal” is used in its conventional sense to refer to the end of an apparatus (or component) that is initially inserted into the patient, or that is closest to the patient during use.

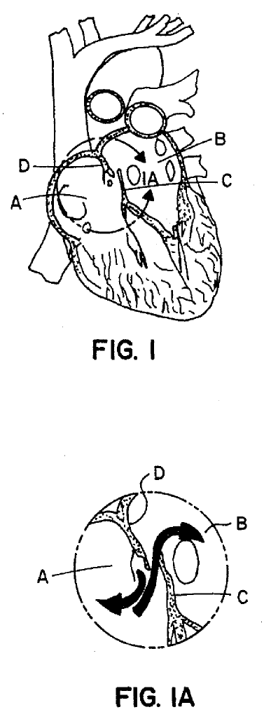

FIG. 1 is an illustration of the internal portions of the heart, showing the right atrium A, left atrium B, atrial septum C that separates the right and left atria, and a tissue flap D that extends along a portion of the atrial septum C. Atrial Septum C and tissue flap D define a channel (shown by the arrows in FIG. 1A) therebetween that comprises the patent foramen ovale. The presence of the PFO undesirably establishes a communication between the atria. This communication allows blood to leak between the chambers, which leakage can result in the migration of unoxygenated blood from the left atrium to mix with the oxygenated blood in the right atrium. Such leakage has been linked to migraine headaches and other maladies described above, such as a stroke or a transient ischemic attack (TIA).

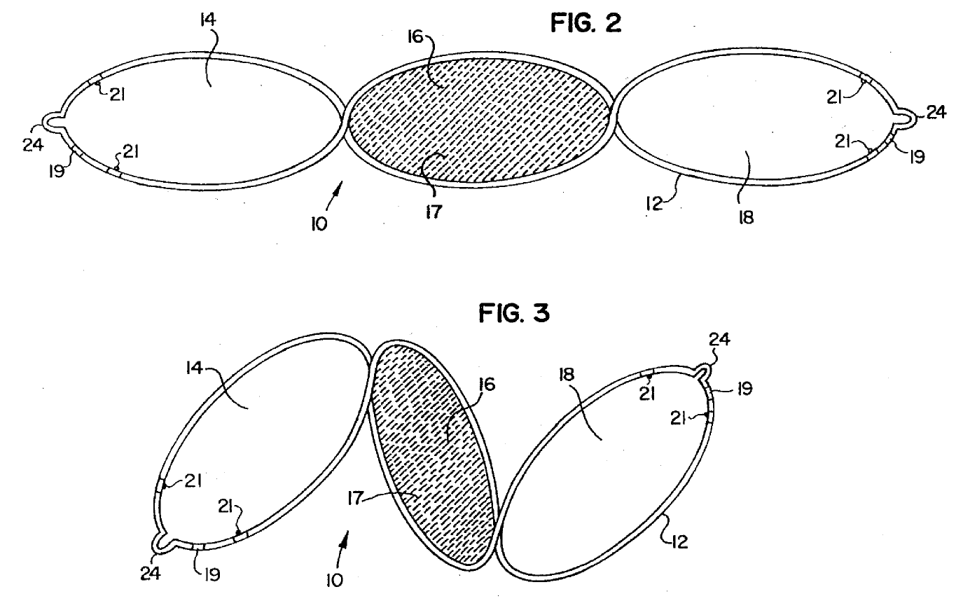

FIG. 2 illustrates an elongated side view of a patent foramen ovale closure apparatus 10, according to one embodiment of the present invention. Apparatus 10 is shown in FIG. 2 in an elongated condition suitable for loading into a deployment apparatus, such as a delivery catheter. Apparatus 10 comprises a generally planar frame member 12 that defines a plurality of segments. Preferably, the segments are in the shape of loops. Most preferably, frame member 12 comprises a continuous wire that is wound or otherwise formed to define three loops 14, 16, 18, as shown in FIG. 2. Alternatively, instead of a plurality of segments formed from a continuous wire, the frame member can comprise a plurality of individual loops or related structures that are sewn, adhered, or otherwise attached in the nature of the loops shown in FIG. 2. Frame member 12 also preferably includes one or more tabs 24 at axial ends thereof, as described herein.

Frame member 12 is preferably formed from a super-elastic material, such as nitinol, a nickel-titanium alloy. Super elastic materials are well known in the medical arts, and routine practitioners are well aware of the capabilities of such materials, and the manner in which they are manipulatable to recover a desired shape. When a frame member is formed from a super elastic wire, the wire is typically manipulated into a desired configuration. Once in the desired configuration, the frame member may be annealed or otherwise treated in a manner to establish a tendency in the material to return to the desired configuration following manipulation to another configuration. With particular reference to frame member 12, a desired configuration will comprise a compressed configuration, such as that shown in FIG. 6. The frame member may be manipulated from the desired configuration to an elongated configuration, such as that shown in FIG. 2. When in the elongated condition, the frame member may be readily loaded into a restraining apparatus, such as a delivery catheter. Upon release of the restraint, e.g., upon withdrawal of the delivery catheter, the elasticity built into the frame member causes it to return to the desired compressed configuration. Those skilled in the medical arts are well aware of the capabilities of super elastic materials, and can readily select an appropriate composition and shape for a particular use.

The characteristics of super elastic compositions, such as nitinol, are well known to those skilled in the art, and further discussion of such characteristics is not necessary to achieve an understanding of the features of the present invention. As an alternative to the use of super elastic materials, other materials having elasticity properties, such as spring tempered wire, may be substituted. Particularly suitable spring tempered wires may be formed from metals or metal alloys, such as the cobalt-chromium-nickel alloys ELGILOY® and INCONEL®. The common property of the compositions useful for forming the frame member is their ability to be arranged in a first shape for loading into a delivery device, and to return to a second shape upon delivery to a target site within the body of the patient. Other biocompatible materials capable of such elasticity, manipulation and/or shape recovery may be substituted for those specifically mentioned hereinabove, all such materials being considered within the scope of the invention.

A covering material 17 is provided along at least one of the loops of the frame member, and preferably, along middle loop 16. Although any number of loops may be covered with covering material 17, it is preferred that at least a portion of the middle loop 16 be covered. The covering material may be formed from one or more components that are suitable for promoting the desired activity of the material. For example, it may be desired to close the foramen ovale by forming a clot within the ovale. In this event, suitable covering materials may include compositions known in the medical arts for promoting clot formation, such as polyester and silk fibers, among others. One particularly suitable covering material in this regard comprises polyester terephthalate (PET) fibers. These fibers are available commercially as DACRON® fibers.

Alternatively, it may be desired to close the foramen ovale by providing a cover material that is capable of growing into the surrounding tissue at each side of the ovale. In this event, suitable covering materials may include growth-promoting compositions. In a preferred embodiment, the growth promoting material is bioremodelable. A bioremodelable material can provide an extracellular matrix that permits, and may even promote, cellular invasion and ingrowth into the material upon implantation. Non-limiting examples of bioremodelable materials include reconstituted or naturally-derived collagenous materials. Preferably, the material is an extracellular matrix material (ECM) possessing biotropic properties, including in certain forms, angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa (SIS), stomach submucosa, urinary bladder submucosa, and uterine submucosa. The submucosa or other ECM material used in the present invention may also exhibit an angiogenic character and thus be effective to induce angiogenesis in a host engrafted with the material.

Suitable bioremodelable material having in vivo angiogenic properties may be identified using a subcutaneous implant model to determine the angiogenic character of a material, as disclosed in C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268. Submucosa or other ECM materials of the present invention can be derived from any suitable organ or other tissue source, usually sources containing connective tissues. The submucosa or other ECM tissue used in the invention is preferably highly purified, for example, as described in U.S. Pat. No. 6,206,931 to Cook et al. Each of the above-identified patents and publications is incorporated herein by reference.

Covering material 17 may also include a bioactive component that induces, directly or indirectly, a cellular response such as a change in cell morphology, proliferation, growth, protein or gene expression. For example, the submucosa material and any other ECM used may also optionally retain growth factors or other bioactive components, such as basic fibroblast growth factor (FGF-2), transforming growth factor beta (TGF-beta), epidermal growth factor (EGF), and/or platelet derived growth factor (PDGF). Further, in addition or as an alternative to the inclusion of native bioactive components, non-native bioactive components such as those synthetically produced by recombinant technology or other methods, may be incorporated into the submucosa or other ECM tissue, including drug substances such as antibiotics or thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient. In addition, covering material 17 can also include additives for promoting both clot formation and tissue growth.

In addition to the foregoing, the covering material can comprise compositions for promoting both clot formation and tissue growth.

As stated, growth and/or clot promoting covering material 17 is preferably provided on at least one of the loops of frame member 12, and most preferably, on the middle loop 16. Typically, covering material 17 is sewn onto frame member 12 when member 12 is in the elongated position as shown in FIG. 2. Alternatively, covering material 17 can be positioned on the loop by other conventional attachment mechanisms, such as laser welding, melting, or gluing with a suitable adhesive.

Preferably, a plurality of barbs 21 or similar anchoring members are provided on the frame member. Barbs, hooks and like structures are commonly applied to insertable structures in the medical arts to enhance securement of the structure to the adjacent tissue, and to stimulate tissue response followed by healing. Those skilled in the art can readily select an appropriate type, number and arrangement of anchoring members for use with the inventive closure apparatus. One preferred arrangement of anchoring members, such as barbs 21, is illustrated in the figures.

If desired, one or more markers, such as radiopaque marker band 19, may be applied to the frame to enhance visibility under conventional medical imaging techniques, such as x-ray fluoroscopy. Although some frame compositions may have sufficient radiopacity such that radiopaque markers are not necessary or helpful, other compositions, such as nitinol, are only weakly radiopaque. With such compositions, the presence of one or more markers will assist visualization. The use of radiopaque markers is well known in the medical arts, and those skilled in the art can readily select and position an appropriate marker or markers for a particular use. Radiopaque markers formed from metals such as tungsten, platinum or gold are particularly preferred for use with frame member 12. Such metals can be conveniently supplied in the form of bands, and can be applied to the apparatus in other well-known manners. Alternatively, instead of applying radiopaque bands to the frame member, radiopaque materials can be incorporated into the matrix of the frame member.

Further details of the inventive apparatus are provided in the following discussion of its use in the closure of a patent foramen ovale. Initially, apparatus 10 is elongated or otherwise manipulated in a generally planar fashion as shown in FIG. 2. Apparatus 10 is then further elongated, or collapsed, e.g., by pulling respective tabs 24 in opposing directions, until apparatus 10 has achieved an outer diameter such that it is receivable within the lumen 32 of a conventional delivery catheter, such as delivery catheter 30 shown in FIG. 7. Delivery catheters for use in delivering medical devices to target sites within the vasculature and other internal bodily sites are well known in the medical arts, and those skilled in the art can readily select an appropriate delivery catheter to deliver apparatus 10 through the vasculature to the site of the patent foramen ovale. For delivery to the interior spaces of the heart, a delivery catheter may be provided with specialized curves on the distal end of the catheter that are oriented to direct the catheter to the desired atrium or ventricle. The delivery catheter must have a length sufficient to extend from an entry vessel, such as the femoral vein in the groin area, through an appropriate pathway, such as the inferior vena cava, into the right atrium or ventricle of the heart, and ultimately into the PFO. Typically, the delivery catheter will have a length of about 80-100 cm. Non-limiting examples of suitable delivery catheters include conventional PTFE sheaths, as well as multi-layered FLEXOR®-type introducer sheaths. FLEXOR® sheaths, typically comprised of a PTFE inner liner, a coiled reinforcement, and an outer jacket formed of a polyether block amide, are commercially available from Cook Incorporated, of Bloomington, Ind.

Delivery catheter 30 may be introduced by conventional means, such as the well-known Seldinger percutaneous entry technique. This technique is commonly used for accessing the right atrium of the heart. In the Seldinger technique, a puncture is made by injecting a needle into the entry vessel. A wire guide is then inserted through a bore in the needle into the vessel, and the needle is thereafter withdrawn. The wire guide is threaded into the right atrium of the heart, and the delivery catheter is threaded over the wire guide into the atrium. Following proper placement of the delivery catheter, the wire guide may be withdrawn in conventional fashion.

Delivery catheter 30 is then advanced through the PFO, such that its distal tip 34 is positioned slightly beyond septum C in left atrium B. Preferably, the positioning of apparatus 10 in delivery catheter 30 is established under fluoroscopy by visualizing the radiopaque marker(s) positioned on the frame member. Upon confirmation of proper placement, withdrawal of the delivery catheter may be commenced. Upon initial withdrawal of the delivery catheter, apparatus loop 18 is deployed from delivery catheter distal tip 34 distal of septum C (left side of heart), e.g., by a conventional pusher apparatus (not shown). Upon further withdrawal of delivery catheter 30 in the proximal direction, and visualization under fluoroscopy, loop 16 is deployed between septum C and flap D. Upon still further withdrawal of delivery catheter 30 in the proximal direction, loop 14 is deployed proximal of flap D.

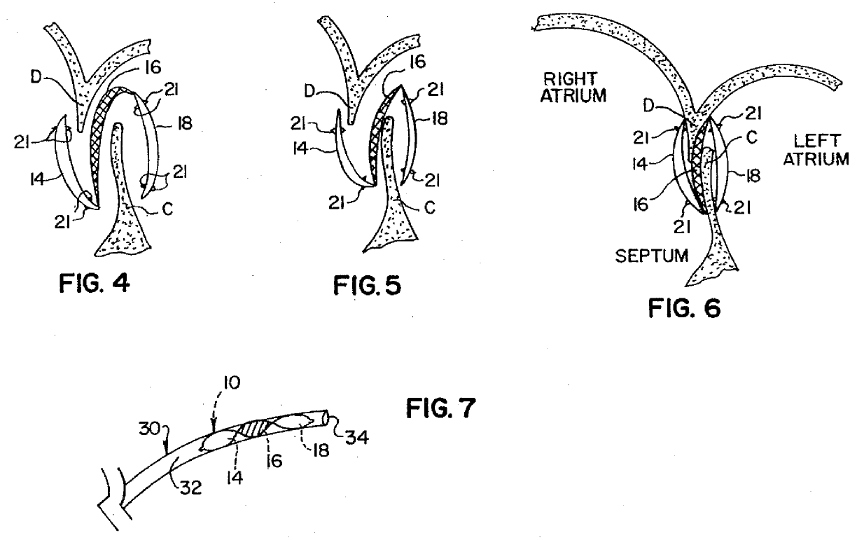

Following deployment of apparatus 10, the delivery catheter is withdrawn. Upon deployment of apparatus 10, the elasticity of the frame member causes the frame member to transform into a closed configuration, as shown in successive FIGS. 4, 5, and 6. Once closure is complete, apparatus 10 achieves the configuration shown in FIG. 6. As apparatus 10 draws septum C and flap D into closely aligned relationship as shown, the clot and/or growth-promoting covering material 17 causes septum C and/or flap D to attach, grow, or otherwise engage with covering material 17. As stated above, frame member 12 also preferably includes barbs 21 or other anchoring members to secure apparatus 10, septum C and flap D in the configuration shown in FIG. 6, thereby further facilitating such growth or adherence.

Although the present invention has been described with reference to its preferred embodiment as an apparatus for closure of a patent foramen ovale, the invention is not so limited. Rather, the inventive apparatus can be extended to the closure of other small channels or passageways encountered within the body of a patient.

While these features have been disclosed in connection with the illustrated preferred embodiments, other embodiments of the invention will be apparent to those skilled in the art that come within the spirit of the invention as defined in the following claims.

What is claimed is:

1. An apparatus for closure of a patent foramen ovale of a patient, the foramen ovale defined by an opening in the heart of the patient between the atrial septum and a tissue flap, the apparatus comprising:
a frame member defining a plurality of frame segments, said frame segments alignable in a generally elongated first position, and movable therefrom to a longitudinally compressed second position, said frame segments structured and arranged such that upon deployment of said apparatus across the foramen ovale in said generally elongated first position and movement therefrom to said compressed second position, a first frame segment is disposed distal of the atrial septum, a second frame segment is disposed intermediate the atrial septum and the tissue flap, and a third frame segment is disposed proximal of the tissue flap, at least one of said frame segments including a cover material disposed at least partially thereover, said cover material capable of promoting biological formation to effect a closure of said foramen ovale.

2. The apparatus of claim 1, wherein said frame member comprises a composition having elasticity sufficient for moving said frame segments from said first position to said second position when said frame member is deployed across the foramen ovate.

3. The apparatus of claim 1, wherein said frame member comprises a super elastic composition or a spring tempered wire capable of self-movement from said first position to said second position.

4. The apparatus of claim 1, wherein said cover material is disposed over at least a portion of said second frame segment.

5. The apparatus of claim 1, wherein the covering material comprises a tissue growth-promoting composition capable of promoting growth with at least one of said atrial septum and tissue flap.

6. The apparatus of claim 5, wherein the tissue growth-promoting composition comprises at least one of a small intestine submucosa and a collagen-based material.

7. The apparatus of claim 1, wherein the covering material comprises a composition capable of promoting clot formation in the foramen ovale.

8. The apparatus of claim 7, wherein the clot-promoting composition comprises a fibrous material.

9. The apparatus of claim 1, wherein the frame member further comprises at least one anchoring member for anchoring the frame member to tissue adjacent the frame member when the frame member is deployed across the foramen ovale.

10. The apparatus of claim 1, further comprising at least one radiopaque marker engaged with the frame member.

11. An apparatus for closure of a patent foramen ovale of a patient, the foramen ovale defined by an opening in the heart of the patient between the atrial septum and a tissue flap, the apparatus comprising:
a frame member defining respective first, second and third frame segments, said frame member being alignable in a generally elongated first position, and movable therefrom to a longitudinally compressed second position, said frame member comprising a composition having elasticity sufficient for moving said frame segments from said first position to said second position when said frame member is deployed across the foramen ovale, said frame segments structured and arranged such that upon deployment of said apparatus across the foramen ovale in said generally elongated first position and movement therefrom to said compressed second position, said first frame segment is disposed distal of the atrial septum, said second frame segment is disposed intermediate the atrial septum and the tissue flap, and said third frame segment is disposed proximal of the tissue flap, said second frame segment including a cover material disposed at least partially thereover, said cover material capable of promoting biological formation to effect a closure of said foramen ovate.

12. The apparatus of claim 11, wherein said frame member comprises a super elastic composition or a spring tempered wire capable of self-movement from said first position to said second position.

13. The apparatus of claim 11, wherein the covering material comprises a tissue growth-promoting composition capable of promoting growth with at least one of said atrial septum and tissue flap.

14. The apparatus of claim 13, wherein the tissue growth-promoting composition comprises at least one of a small intestine submucosa and a collagen-based material.

15. The apparatus of claim 11, wherein the covering material comprises a fibrous material capable of promoting clot formation in the foramen ovale.

16. A method for closure of a patent foramen ovale, the patent foramen ovale defined by an opening in the heart of the patient between the atrial septum and a tissue flap, the method comprising:
providing a closure apparatus for said foramen ovale, said closure apparatus comprising a frame member defining a plurality of frame segments, said frame segments alignable in a generally elongated first position, and movable therefrom to a longitudinally compressed second position, said frame segments structured and positioned such that upon deployment of said apparatus across said patent foramen ovale a first frame segment is disposed distal of the atrial septum, a second frame segment is disposed intermediate the atrial septum and the tissue flap, and a third frame segment is disposed proximal of the tissue flap, at least one of said frame segments including a cover material disposed at least partially thereover, said cover material capable of promoting biological formation to effect a closure of said foramen ovale;
loading said closure apparatus into a delivery sheath in said elongated position;
percutaneously introducing said delivery sheath and loaded closure apparatus into the right atrium of the heart of a patient;
advancing the delivery sheath and loaded closure apparatus such that a distal tip of the delivery sheath is positioned distal of the atrial septum, and deploying said first frame segment from the delivery sheath;
withdrawing the delivery sheath such that the distal tip is positioned intermediate the atrial septum and the tissue flap, and deploying said second frame segment from the delivery sheath; and
further withdrawing the delivery sheath such that the distal tip is positioned proximal of the tissue flap, and deploying said third frame segment from the delivery sheath.

17. The method of claim 16, further comprising the step of verifying the placement of the closure apparatus across the foramen ovale prior to deployment of at least one of the frame segments.

18. The method of claim 16, wherein the frame member comprises a super elastic composition.

19. The method of claim 16, wherein the cover material comprises a tissue growth-promoting composition capable of promoting growth with at least one of said atrial septum and tissue flap.

20. The method of claim 16, wherein the cover material comprises a composition capable of promoting clot formation in said foramen ovale.
